(19) United States
(12) Patent Application Publication
(10) Pub. No.: US 2025/0345543 A1
(43) Pub. Date: Nov. 13, 2025

(54) SPEAKING VALVE, CLOSURE MEANS FOR A SPEAKING VALVE, AND METHOD

(71) Applicant: ANDREAS FAHL MEDIZINTECHNIK-VERTRIEB GMBH, Köln (DE)

(72) Inventor: Andreas FAHL, Köln (DE)

(21) Appl. No.: 17/785,684

(22) PCT Filed: Dec. 15, 2020
(86) PCT No.: PCT/EP2020/086202

§ 371 Date: Jul. 24, 2025

(30) Foreign Application Priority Data
Dec. 16, 2019 (DE) 102019134507.3

(51) Int. Cl. A61M 16/04

(52) U.S. Cl. CPC A61M 16/0468

(57) ABSTRACT

A speaking valve comprising at least a housing and a closing means is proposed. The housing comprises an insertion part and a cover part with a breathing opening, wherein the cover part can be arranged on the insertion part, wherein the closing means comprises an outer ring, a sealing means and at least one mounting, wherein the sealing means is disposed on the outer ring by means of the mounting, wherein the cover part forms a valve seat for abutment of the sealing means and the breathing opening can be closed by means of the sealing means.

### CROSS-REFERENCE TO RELATED APPLICATIONS

This application is the U.S. national stage of PCT/EP2020/086202 filed Dec. 15, 2020, which claims priority of German patent application 10 2019 134 507.3 filed Dec. 16, 2019, both of which are hereby incorporated by reference in their entirety.

### FIELD OF THE INVENTION

The invention relates to a speaking valve at least comprising a housing and a closing means, a closing means for a speaking valve, and a method for adjusting a closing pressure.

### BACKGROUND OF THE INVENTION

Speaking valves are sufficiently known from the prior art. For example, DE 10 2014 002 063 A1 describes a speaking valve with a cover part, an actuating means, a housing part and a filter, wherein the housing part provides a valve seat arranged proximally therein, wherein the actuating means comprises a closing part and wherein the closing part cooperates with the valve seat when the speaking valve is transferred from an open position to a closed position, wherein the cover part is at least partially formed of an elastic material, wherein an at least partial deformation of at least a region of the cover part is effected by a transfer of the speaking valve from the open position to the closed position, and wherein the filter is arranged at the actuating means such that the filter can substantially follow the movement of the actuating means.

A disadvantage of the known speaking valves is that they must be manually transferred from an open position to a closed position in order to initiate a speaking process. This means that a naturally appearing speaking action is not possible and the user is not able to gesticulate freely.

It is therefore the object of the invention to improve the speaking valves known from the prior art. In particular, the object is to provide a speaking valve with which a speaking process can be initiated without the use of the hands.

The object is solved according to the invention by means of a speaking valve at least comprising a housing and a closing means, wherein the housing comprises an insertion part and a cover part with a breathing opening, wherein the cover part can be arranged on the insertion part, wherein the closing means comprises an outer ring, a sealing means and at least one mounting, wherein the sealing means is arranged on the outer ring by means of the mounting, wherein the cover part forms a valve seat for the abutment of the sealing means and the breathing opening can be closed by means of the sealing means.

Furthermore, the object is solved according to the invention by means of a closing means for a speaking valve comprising an outer ring, a sealing means and at least one mounting, wherein the sealing means is arranged on the outer ring by means of the mounting.

Furthermore, the object is solved according to the invention by means of a method for adjusting a closing pressure of a speaking valve, comprising a housing and a closing means, the housing comprising an insertion part and a cover part with a breathing opening and a valve seat, the cover part being disposable on the insertion part, wherein the closing means comprises an outer ring, a sealing means and at least one mounting, wherein the sealing means is arranged on the outer ring by means of the mounting, wherein a distance between the sealing means and the valve seat is varied by turning the cover part on the insertion part.

### SUMMARY OF THE INVENTION

A speaking valve at least comprising a housing and a closing means is proposed. The housing comprises an insertion part and a cover part with a breathing opening, wherein the cover part can be arranged on the insertion part, wherein the closing means comprises an outer ring, a sealing means and at least one mounting, wherein the sealing means is arranged on the outer ring by means of the mounting, wherein the cover part forms a valve seat for the abutment of the sealing means and the breathing opening can be closed by means of the sealing means.

During surgical interventions in the upper respiratory tract, it may be necessary to create an artificial breathing opening (tracheostoma) in the trachea so that air can be breathed directly into the lungs, bypassing the oral cavity and larynx. For example, in the case of laryngectomized persons, i.e. persons without a larynx, in whom the larynx has been surgically removed, the tracheostoma must be kept permanently open and stabilized, for which purpose tracheal cannulae in particular, usually consisting of an outer and an inner cannula, are inserted into the tracheostoma. But the use of so-called “tracheostoma buttons” is also possible, especially for people who no longer need a tracheostomy cannula.

Finally, filter systems can also be inserted into the tracheostoma for both tracheostomized and laryngectomized patients. Such filter systems consist in particular of a patch with an inserted filter in a housing or of a usually self-adhesive base plate-generally made of plastic, in which filters of various types can be inserted in a housing. Filter systems used for laryngotracheal devices such as tracheal cannulas, shunt valves, tracheostomy buttons and filter systems with patch or base plate include generic heat and moisture exchangers, also called “artificial nose”. These serve to recreate the regulatory mechanisms for heating and humidifying the air breathed, which are lacking in laryngectomized and also tracheotomized people, and to avoid bringing the trachea into contact with increasingly dry, cold and unfiltered air. This is because the irritation caused by this results in increased mucus production with the subsequent risk of clogging. The inhaled air is moistened, warmed and filtered at the same time by heat and moisture exchangers. This largely prevents the aforementioned formation of cloggings. Wearing the artificial nose regularly helps, especially in cases of heavy secretion, because moistening the mucous membranes in the trachea reduces secretion production.

Heat and moisture exchangers for laryngectomized and tracheostomized patients have a filter material-usually made of paper or foam-through which the inhaled and exhaled air is passed. During exhalation, the filter material retains moisture, which is then transported into the trachea during inhalation. Heat and moisture exchangers known from the prior art are available in a large number of different designs for different adapters. A universal adapter according to DIN EN ISO 5356-1 has a diameter of about 15 mm, but other adapters with a diameter of about 22 mm are also known from the prior art.

Another aid for laryngectomized patients are voice prostheses, which are used to restore the voice after a laryngectomy. The voice can be restored by surgical means. The most important aid in this process is a voice prosthesis, also known as a “shunt valve”. By means of the voice prosthesis, functions of the removed larynx can be replaced. On the one hand, the use of the voice prosthesis enables an air supply from the lungs via the esophagus into the pharynx. The exhaled air is used for speaking. On the other hand, the voice prosthesis seals the connection from the esophagus to the trachea when swallowing food and drinks, protecting the user of the voice prosthesis from accidentally swallowing food.

Once the wounds have healed after the surgical procedure, people are unable to speak in their normal state. To enable speaking, when a voice prosthesis is used or the larynx is still present in tracheotomized people, the air outlet at the neck must be closed so that the air can pass through the voice prosthesis or the larynx, respectively. Speaking valves are used for this purpose.

The proposed speaking valve has the advantage that it closes automatically at the intention of speaking. This is made possible in particular by the fact that an exhalation pressure is naturally increased to initiate the speaking process. The increased pressure displaces the sealant in the speaking valve in such a way that it can come into contact with the valve seat. This closes the breathing opening and the exhaled air can be passed through the shunt valve or the larynx, which may still be present.

The speaking valve comprises the housing. The housing has a first side and an opposing second side. The first side of the housing is preferably distally disposable so as to face away from a body surface of a user during use. The second side of the housing is preferably proximally disposable so as to face the user's body surface during use.

The housing further comprises the insertion part. In one embodiment, the insertion part comprises a first side and a second side opposite the first side. The first side of the insertion part is preferably disposable distally so as to face away from a body surface of a user during use. The second side of the insertion part is preferably proximally disposable so as to face the user's body surface during use.

In one embodiment, the insertion part comprises an insertion portion. The insertion portion is preferably associated with the second side. Further, the insertion portion comprises a connector for connection to a laryngotracheal device. In a further embodiment, the insertion portion is configured as a connector. In a further preferred embodiment, the insertion portion comprises an outer diameter of about 10 mm to about 25 mm, preferably about 15 mm or about 22 mm.

If, within the scope of the invention, the term “about” is used in connection with values or ranges of values, this is to be understood as a range of tolerance which the person skilled in the art considers to be usual in this field, in particular a range of tolerance of ±20%, preferably ±10%, further preferably ±5% is provided.

Insofar as different value ranges, for example preferred and further preferred value ranges, are indicated in the present invention, the lower limits and the upper limits of the different value ranges can be combined with each other.

For example, a laryngotracheal device may be a tracheostomy tube and/or a tracheostomy patch.

Exemplary enumerations are not to be regarded as conclusive in the sense of the invention, but may be supplemented within the scope of general technical knowledge.

In a further embodiment, it is provided that the insertion part comprises a threaded portion. The threaded portion is preferably associated with a first side of the insertion part. Preferably, the threaded portion comprises a height extending in a longitudinal direction of the insertion part. Further preferably, the threaded portion comprises at least one, preferably exactly one, thread. Further preferably, the threaded portion is configured as an external thread. In one embodiment, the threaded portion comprises a metric thread pitch of about 0.5 to about 2 times, preferably about 0.7 to about one times, more preferably about 0.75 times a height of the threaded portion.

In the sense of the invention, a metric thread pitch is the distance traveled by a complete turn of the cover part on the housing in the longitudinal direction. In particular, the metric thread pitch is the distance between two thread tips of a thread immediately following each other in the longitudinal direction.

In a further embodiment, the insertion part comprises a filter recess. The filter recess is in particular a space at least partially enclosed by an inner wall of the insertion part. In particular, the filter recess is a recess for receiving a filter, in particular described below, in particular in the insertion part. Further preferably, the insertion part is substantially designed as a cylinder. Further preferably, the insertion part comprises a cylindrical recess which can be addressed as a filter recess. In particular, the filter recess is at least radially defined by the substantially cylindrical inner wall. The cylindrical inner wall is in particular an inner surface of the insertion part. In another embodiment, the cylindrical inner wall comprises a filter detent. In one embodiment, the speaking valve comprises the filter disposed in the insertion part, the insertion part comprising the filter detent for retaining the filter in the insertion part. In particular, the filter detent is configured such that a filter arranged in the insertion part is substantially not moved in the direction of the first side, further preferably does not come into contact with the closing means, further preferably does not hinder the closing means in its function when a pressure, preferably a breathing pressure, acts on the filter. The filter detent can be designed, for example, as a radially inwardly pronounced accumulation of material. In a further embodiment, the filter detent comprises at least one hook. In a further embodiment, the filter recess is defined towards the second side by a respiration shield. The respiration shield preferably prevents inhalation by the user of a filter located in the insertion part. In particular, in one embodiment, the respiration shield comprises at least one strut or grid.

The term “substantially” indicates a range of tolerance which is acceptable to the person skilled in the art from an economic and technical point of view, so that the corresponding feature is still recognizable or realized as such.

In a further embodiment, it is provided that the insertion part comprises a stop. In particular, the stop can be used to limit insertion of the insertion portion into a laryngotracheal device. The stop can, for example, be designed as an at least partially circumferential collar. In a further embodiment, the stop is configured as at least one accumulation of material on the outer circumference of the insertion part. In particular, the stop is arranged on the first side of the insertion portion. Further preferably, the stop is arranged between the threaded portion and the insertion part.

In an alternative embodiment, it is provided that the cover part can be arranged on the insertion part by means of a bayonet catch.

In a further embodiment, the housing, more preferably the insertion part or the cover part, comprises a closing means receptacle. In one embodiment, the closing means receptacle is associated in particular with the first side of the insertion part. In one embodiment, the closing means receptacle is associated in particular with the second side of the cover part. Preferably, the closing means receptacle is arranged on or formed by the cylindrical inner wall of the insertion part or the inner wall of the cover part. In one embodiment, it is provided that the closing means receptacle is configured as an at least partially circumferential, preferably fully circumferential notch, preferably in the cylindrical inner wall of the insertion part or the inner wall of the cover part. In a further embodiment, it is provided that the closing means receptacle comprises latching means, preferably comprising latching means formed as an accumulation of material. In a further embodiment, it is provided that the closing means receptacle comprises at least one at least partially circumferential bead, in particular on the cylindrical inner wall of the insertion part or the inner wall of the cover part. Further preferably, two at least partially circumferential beads spaced from each other in the longitudinal direction of the housing are provided, which can be addressed as a closing means receptacle. Preferably, the closing means receptacle is designed to substantially fix the closing means in or on the housing, preferably to keep it spaced from the filter, further preferably, in particular in the case of an exhalation pressure, to limit a movement of the closing means in the direction of the first side.

The housing further comprises the cover. In particular, the cover part is hood-shaped or dome-shaped. In a further embodiment, the cover part is in the form of a cap nut. In particular, the cover part comprises a first side and a second side opposite the first side. Preferably, the first side is disposable distally and the second side is disposable proximally when the speaking valve is used.

In particular, the cover part comprises a shell. Further preferably, the cover part comprises an inner wall, which is preferably an inner surface of the shell. Further preferably, the cover part comprises an annular wall. The annular wall is preferably associated with the first side of the cover part. Furthermore, a surface of the annular wall facing the first side comprises a surface vector directed substantially in the direction of the first side. In particular, a breathing opening is formed by the annular wall. Preferably, it is provided that the annular wall is adjacent to or merges into the shell. In particular, a transition region between the shell and the ring wall is formed as a chamfer and/or radius. In a further embodiment, it is provided that the annular wall forms a valve seat, in particular on the second side. The valve seat is in particular a surface on the second side of the annular wall which comprises a surface vector which is directed in the direction of the second side. In particular, the valve seat is formed in such a way that it can cooperate with the closing part.

The cover part comprises the cover thread. In particular, it is provided that the cover thread is designed as an internal thread. In particular, it is provided that the cover thread, which is preferably designed as an internal thread, is arranged on the inner wall of the cover part. In one embodiment, the inner wall of the cover part comprises the cover thread. In another embodiment, the cover thread comprises a metric thread pitch of about 0.5 to about 2 times, preferably about 0.7 to about 1 times, more preferably about 0.75 times a height of the cover thread.

Preferably, the thread run of the insertion part and the cover thread are matched to each other. In one embodiment, the thread and the cover thread are provided with about 0.5 turns to about 2 turns, preferably about 0.7 turns to about one turn, more preferably about 0.75 turns. In one embodiment, it is provided that the thread and the cover thread is a metric thread pitch of about 0.5 to about 2, preferably about 0.7 to about 1, more preferably about 0.75 times a height of the thread portion and/or the cover thread.

In one embodiment, the insertion part comprises a threaded portion having a thread and the cover part comprises a cover thread, wherein the thread of the insertion part and the cover thread are configured to correspond with each other. In a further embodiment, it is provided that the cover thread and/or the threaded portion of the insertion part have a stop which preferably limits turning of the cover part on the insertion part. The stop may, for example, be associated with an end of the cover thread and/or an end of the threaded portion. In one embodiment, the stop can be overcome by means of a defined force or a defined moment. For example, the cover part can be screwed onto the insertion part by applying an increased torque to the insertion part, preferably until the stop is overcome and, more preferably, the cover part is snapped onto the insertion part. In one embodiment, for example, it is provided that the cover part can be screwed on with a lower torque than it can be unscrewed. In particular, the stop of the cover part and/or of the insertion part comprises at least one inclined plane. In particular, a geometry of the stop of the cover part and/or of the insertion part is provided, in which a flatter inclined plane must be overcome during screwing on than during unscrewing. In a preferred embodiment, the cover part snaps onto the insertion part with an audible noise after overcoming the stop. The stop is preferably provided to ensure accidental disassembly when the cover part is turned on the insertion part.

In one embodiment, it is provided that the speaking valve comprises the filter. The filter is preferably provided in an embodiment in which the speaking valve is also configured as a heat-moisture exchanger. Preferably, the filter can store heat and moisture from the exhaled air and release it to the inhaled air upon inhalation. In one embodiment, the filter comprises a first side and a second side opposite the first side. Preferably, the first side is disposable distally and the second side is disposable proximally when the speaking valve is in use. In one embodiment, the filter is cylindrical. In particular, the filter can be arranged in the filter recess of the insertion part. In one embodiment, the filter comprises at least one material selected from a group comprising paper, polyurethane, polyethylene, polypropylene and/or biopolymers.

The speaking valve comprises the closing means. The closing means comprises a first side and a second side opposite to the first side. Preferably, when the speaking valve is in use, the first side is disposable distally and the second side is disposable proximally.

In one embodiment, it is provided that the closing means can be arranged at least partially in an at least partially circumferential closing means receptacle of the housing. In particular, a form-fitting connection can be produced between the closing means receptacle and the closing means. In particular, the closing means is snapped into the closing means receptacle.

The closing means is intended in particular to close the breathing opening of the cover part. Preferably, the closing means allows the breathing opening to be closed by means of the closing means at a defined exhalation pressure, in particular without manual intervention by the user.

The closing means comprises the outer ring. Preferably, the outer ring is completely closed. In particular, the outer ring comprises an annular surface. In particular, the ring shell surface is a radial surface of the outer ring. In one embodiment, the ring shell surface comprises at least one at least partially circumferential material accumulation. In particular, the material accumulation is in the form of a bead. In a further embodiment, it is provided that the material accumulation is designed in such a way that it can cooperate with the closing means receptacle, in particular can be snapped into the latter.

The closing means comprises the sealing means. The sealing means is arranged in particular centrally, preferably concentrically, in the outer ring. Further preferably, the sealing means is substantially circular in shape. In one embodiment, the sealing means is designed to be closed over the entire surface, in particular the sealing means is designed as a circular disc. Preferably, the sealing means has a larger radius than the breathing opening of the cover part. In a further embodiment, the sealing means is configured such that it can be placed at least partially on the valve seat of the cover part. Preferably, the sealing means cooperates with the valve seat to close the breathing opening.

Preferably, the sealing means is arranged on the outer ring by means of at least one mounting. In particular, the at least one mounting is arranged between the outer ring and the sealing means. Further preferably, the closing means comprises about one to about 12, preferably about 3 to about 8, more preferably about 3 to about 4, more preferably exactly 4 mountings. In one embodiment, it is provided that the at least one mounting is web-shaped. In particular, the at least one mounting can be slotted or unslotted. If the mounting is slotted, it comprises in particular centrally one or more recesses which are completely continuous in particular from the first side to the second side and which further preferably extend in the longitudinal direction of the mounting. In a further embodiment, it is provided that the at least one mounting is materially bonded to the outer ring. In a further embodiment, it is provided that the at least one mounting is materially connected to the sealing means. In a further embodiment, it is provided that the mounting is glued, molded or otherwise attached to the sealing means and/or to the outer ring.

In one embodiment, it is provided that by means of the mounting the sealing means is rubber-elastically mounted on the outer ring. Preferably, the closing means, more preferably the outer ring, the sealing means and/or the at least one mounting comprises at least one material selected from a group at least comprising thermoplastic elastomers, silicone and/or rubber.

Advantageously, the sealing means is arranged in the housing in such a way that a defined exhalation pressure presses the sealing means towards the first side, in particular towards the valve seat. As a rule, the exhalation pressure is greater when a speaking process is initiated than during normal breathing. In particular, when a speaking process is initiated or breathing pressure is increased, the sealing means is placed in such a way that it closes the breathing opening. The exhaled air can thus be directed, for example, through a voice prosthesis into the esophagus or through the larynx, and thus the user is enabled to speak without manual actuation of the speaking valve. In particular, the speaking valve is adjustable so that when exhalation pressure is applied to initiate a speaking process, the sealing means is in sealing contact with the valve seat and the breathing opening is closed. Since the exhalation pressure for speaking is individual for each person and, depending on the user's actuation, the exhalation pressure for normal breathing may also vary, the proposed speaking valve can be advantageously adjusted by turning the cover part on the insertion part.

In one embodiment, it is provided that a distance of the sealing means from the valve seat can be varied by turning the cover part on the insertion part. By turning the cover part on the insertion part, the cover part is further screwed on or off so that a distance between the valve seat and the sealing means in a neutral position can be varied. The neutral position of the sealing means is particularly given when there is no exhalation pressure or inhalation pressure in the speaking valve and the sealing means is held centrally in the outer ring. If, for example, the cover part is screwed on completely, the distance between the valve seat and the sealing means is small. In this case, only a low exhalation pressure is required to move the sealing means sufficiently far out of the neutral position so that it is in contact with the valve seat. If, for example, the cover part is unscrewed almost completely, in particular as far as a stop, increased exhalation pressure is required to move the sealing means sufficiently far out of the neutral position so that it rests against the valve seat. If necessary, the cover part can be unscrewed to such an extent, and in particular the distance between the valve seat and the sealing means in the neutral position can be adjusted in such a way that no exhalation pressure exerted by the user can bring the sealing means into contact with the valve seat. This may be particularly desirable for sports activities. If speaking is then to be enabled again, the user can continue to screw the cover part onto the insertion part until the distance between the valve seat and the sealing means in the neutral position is sufficiently close to initiate a speaking process.

In an exemplary embodiment, the speaking valve comprises a housing having a cover part and an insertion part. The cover part comprises a first side and a second side opposite thereto. The first side is distally disposable in use of the speaking valve. The second side is disposable proximally in use of the speaking valve. The cover part comprises a shell comprising a cover thread on its inner side. The cover thread is exemplarily a single-threaded internal thread with a pitch of about 0.75 of the height of the cover thread. Furthermore, the cover part comprises, by way of example, an annular wall which is connected to the shell via a transition in the form of a chamfer. The annular wall centrally comprises a breathing opening. Furthermore, the annular wall comprises a valve seat which is arranged on the second side of the annular wall. The valve seat surrounds the breathing opening.

The exemplary housing further comprises an insertion part. This comprises a first side and a second side opposite thereto. The first side is distally disposable when the speaking valve is in use. The second side is disposable proximally during use of the speaking valve. The insertion part comprises an insertion portion and a threaded portion. The threaded portion comprises a thread having a height and a metric thread pitch of, by way of example, about 0.75 times the height. Associated with the second side, the insertion part comprises an insertion portion which assumes the function of a 15 mm or 22 mm connector. With the insertion portion, the speaking valve can be connected to an exemplary tracheostomy aid which is not shown. A stop is arranged between the insertion portion and the threaded portion, which limits both screwing of the cover part onto the threaded portion and insertion of the insertion portion into a tracheostomy aid.

The exemplary insertion part comprises a filter recess in which a filter can be received. The filter recess is defined towards the second side by a respiration shield. The respiration shield comprises, by way of example, a number of webs which prevent inhalation of the filter. Associated with the first side is a closing means receptacle arranged in an inner wall defining the filter recess, which is designed as a circumferential notch. A closing means can snap into the closing means receptacle. For this purpose, the closing means comprises a bead on an outer ring, for example.

In the exemplary embodiment, if the cover part is screwed onto the insertion part, the threaded portion engages in the cover thread. By turning the cover part on the insertion part, a distance between the valve seat and the closing means, which is locked in the insertion part, can consequently be varied. Exemplarily, the threaded portion and/or the cover thread may include a stop that limits turning of the cover part on the insertion part and, in particular, prevents accidental separation of the cover part from the insertion part.

The exemplary closing means comprises a first side and a second side. The first side is disposable distally when the speaking valve is in use. The second side is disposable proximally during use of the speaking valve. The closing means comprises an outer ring which comprises a circumferential bead on its ring shell. A sealing means is arranged concentrically in the outer ring in a neutral position. The sealing means is held by exemplary four mountings. In further exemplary embodiments, more or fewer mountings may also be provided.

In the use of the exemplary speaking valve, pressure is built up from the second side during exhalation, which de-places the sealant towards the first side. This de-placement is greater when the user attempts to speak, as naturally the exhalation pressure is greater when speaking. When the cover part is screwed onto the insertion part to such an extent that the sealant thereby comes into contact with the valve seat, the speaking valve is closed and the exhaled air can flow through the user's larynx or a shunt valve to allow the user to speak.

Furthermore, a closing means for a speaking valve described above is proposed. The closing means comprises an outer ring, a sealing means and at least one mounting, wherein the sealing means is arranged on the outer ring by means of the mounting. In particular, the closing means may be configured as described above.

The closing means comprises a first side and a second side opposite the first side. Preferably, the first side is disposable distally and the second side is disposable proximally when the speaking valve is in use.

In particular, the closing means is intended to close the breathing opening of the cover part described above. Preferably, the closing means allows the breathing opening to be closed by means of the closing means at a defined exhalation pressure, in particular without manual intervention by the user.

The closing means comprises the outer ring. Preferably, the outer ring is completely closed. In particular, the outer ring comprises an annular surface. The ring shell surface is in particular a radial surface of the outer ring. In one embodiment, the ring shell surface comprises at least one at least partially circumferential material accumulation. In particular, the material accumulation is in the form of a bead. In a further embodiment, it is provided that the material accumulation is designed in such a way that it can cooperate with the closing means receptacle, in particular can be snapped into it.

The closing means comprises the sealing means. The sealing means is in particular disposed centrally, preferably concentrically, in the outer ring. Further preferably, the sealing means is substantially circular in shape. In one embodiment, the sealing means is designed to be closed over the entire surface, in particular the sealing means is designed as a circular disc. Preferably, the sealing means has a larger radius than the breathing opening of the cover part. In a further embodiment, the sealing means is configured such that it can be placed at least partially on the valve seat of the cover part. Preferably, the sealing means cooperates with the valve seat to close the breathing opening.

Preferably, the sealing means is arranged on the outer ring by means of at least one mounting. In particular, the at least one mounting is arranged between the outer ring and the sealing means. Further preferably, the closing means comprises from about one to about 12, preferably from about 3 to about 8, more preferably from about 3 to about 4, more preferably exactly 4 mountings. In one embodiment, it is provided that the at least one mounting is web-shaped. In particular, the at least one mounting can be slotted or unslotted. If the mounting is slotted, it comprises in particular centrally one or more recesses that are completely continuous, in particular from the first side to the second side, and which further preferably extend in the longitudinal direction of the mounting. In a further embodiment, it is provided that the at least one mounting is materially bonded to the outer ring. In a further embodiment, it is provided that the at least one mounting is materially connected to the sealing means. In a further embodiment, it is provided that the mounting is glued, molded, or otherwise secured to the sealing means and/or to the outer ring.

In one embodiment, it is provided that by means of the mounting, the sealing means is rubber-elastically mounted on the outer ring. Preferably, the closing means, more preferably the outer ring, the sealing means and/or the at least one mounting comprises at least one material selected from a group at least comprising thermoplastic elastomers, silicone and/or rubber.

Advantageously, the sealing means is arranged in the housing in such a way that a defined exhalation pressure presses the sealing means towards the first side, in particular towards the valve seat. As a rule, the exhalation pressure is greater when a speaking process is initiated than during normal breathing. In particular, the sealing means can be de-placed during the initiation of a speaking process or an increased breathing pressure.

An exemplary closing means comprises a first side and a second side. The first side is disposable distally when the speaking valve is in use. The second side is disposable proximally during use of the speaking valve. The closing means comprises an outer ring which comprises a circumferential bead on its ring shell. A sealing means is arranged concentrically in the outer ring in a neutral position. The sealing means is held by exemplary four mountings. In further exemplary embodiments, more or fewer mountings may also be provided.

Furthermore, a method for adjusting a closing pressure of a speaking valve described above is proposed. The speaking valve comprises a housing and a closing means, wherein the housing comprises an insertion part and a cover part with a breathing opening and a valve seat. The cover part can be arranged on the insertion part. The closing means comprises an outer ring, a sealing means and at least one mounting, whereby the sealing means is arranged on the outer ring by means of the mounting. By turning the cover part on the insertion part, a distance between the sealing means and the valve seat is varied.

In an exemplary embodiment of the method, the cover part is turned at least to such an extent that a distance between the closure means, in particular the sealing means, is such that an exhalation pressure does not bring the sealing means into contact against the valve seat. This is particularly advantageous if the user does not want to speak and in particular wants to prevent air from flowing through the shunt valve, for example during sports.

In a further exemplary embodiment of the method, the cover part is turned at least to such an extent that a distance between the closing means, in particular the sealing means, is such that an exhalation pressure brings the sealing means into contact against the valve seat. In particular, the user can screw the cover part onto the insertion part just enough so that a deplacement of the sealing means during exhalation with an intention to speak is sufficient to come into contact with the valve seat. Preferably, the user tries to speak and screws the cover part onto the insertion part until the breathing opening is closed. If the breathing opening is closed unintentionally, the user preferably unscrews the cover part again until this no longer occurs during normal breathing in the given situation.

### BRIEF DESCRIPTION OF THE DRAWINGS

Further advantageous embodiments are shown in the following figures. However, the further embodiments shown there are not to be interpreted restrictively; rather, the features described there can be combined with one another and with the features described above to form further embodiments. Furthermore, it should be noted that the reference signs given in the figure description do not limit the scope of protection of the present invention, but merely refer to the embodiments shown in the figures. Identical parts or parts with the same function have the same reference signs in the following. They show:

FIG. 1 an exploded view of a speaking valve; and

FIG. 2 a closing means in a detailed view.

### DETAILED DESCRIPTION OF THE PREFERRED EMBODIMENTS

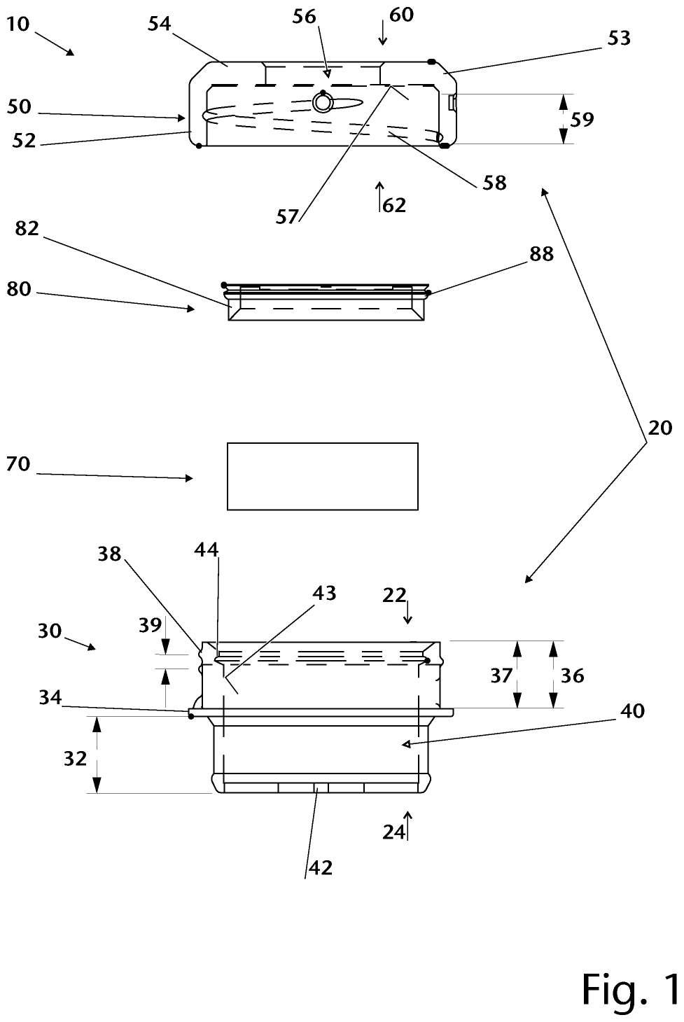

FIG. 1 shows an exploded view of a speaking valve 10, which comprises a housing 20 having a cover part 50 and an insertion part 30. The cover part 50 comprises a first side 60 and a second side 62 opposite the first side 60. The first side 60 is distally disposable in use of the speaking valve 10. The second side 62 is proximally disposable in use of the speaking valve 10. The cover part 50 comprises a shell 52 that comprises a cover thread 58 on its inner surface. The cover thread 58 is a single-start female thread having a pitch of about 0.75 of the height 59 of the cover thread 58. Furthermore, the cover part 50 comprises an annular wall 54, which is connected to the shell via a transition 53 configured as a chamfer. The annular wall 54 centrally comprises a breathing opening 56. Furthermore, the annular wall 54 comprises a valve seat 57, which is arranged on the second side 62 of the annular wall 54. The valve seat surrounds the breathing opening 56.

The housing 20 further comprises the insertion part 30, which comprises a first side 22 and a second side 24 opposite the first side 22. The first side 22 is disposable distally when the speaking valve 10 is in use. The second side 24 is disposable proximally during use of the speaking valve 10. The insertion part 30 comprises an insertion portion 32 and a threaded portion 36. The threaded portion 36 comprises a thread 38 having a height 37 and a metric thread pitch 39 of about 0.75 times the height 37. Associated with the second side 24, the insertion part 30 comprises an insertion portion 32 that performs the function of a 15 mm or 22 mm connector. With the insertion portion 32, the speaking valve 10 can be connected to a tracheostomy aid that is not shown. A stop 34 is arranged between the insertion portion 32 and the threaded portion 36, which limits both screwing of the cover part 50 onto the threaded portion 36 and insertion of the insertion portion 32 into a tracheostomy aid.

The insertion part 30 comprises a filter recess 40 into which a filter 70 can be received. The filter recess 40 is defined towards the second side 24 by a respiration shield 42. The respiration shield 42 comprises a number of webs that prevent inhalation of the filter 70. Associated with the first side 22, a closing means receptacle 44 is disposed in an inner wall 43 defining the filter recess 40 and is configured as a circumferential notch. A closing means 80 can snap into the closing means receptacle 44. For this purpose, the closing means comprises a bead 88 on an outer ring 80.

When the cover part 50 is screwed onto the insertion part 30, the threaded portion 36 engages the cover thread 58. By turning the cover part 50 on the insertion part 30, a distance of the valve seat 57 to the closing means 80, which is locked in the insertion part 30, can consequently be varied. As mentioned in the general description, the threaded portion 36 and/or the cover thread 58 may include a stop that limits turning of the cover part 50 on the insertion part 30 and, in particular, prevents accidental separation of the cover part 50 from the insertion part 30. Embodiments of the stop are described above by way of example.

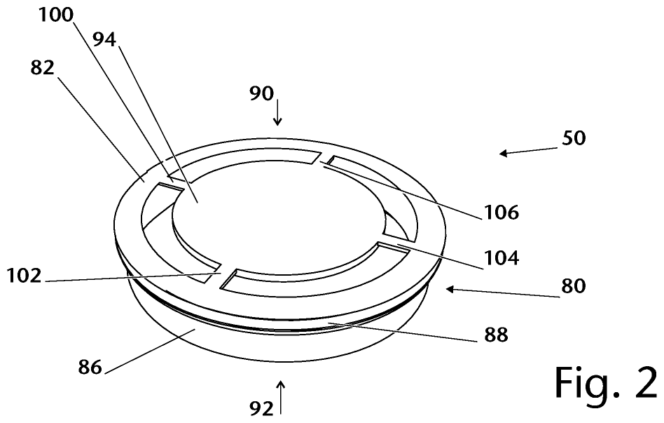

FIG. 2 shows a detailed view of the closing means 80 of FIG. 1. The closing means 80 comprises a first side 90 and a second side 92. The first side 90 is disposable distally during use of the speaking valve 10. The second side 92 is disposable proximally during use of the speaking valve 10. The closing means 80 comprises an outer ring 82 which comprises a circumferential bead 88 on its annular shell 86. A sealing means 94 is concentrically disposed in the outer ring 82 in a neutral position. The sealing means 94 is held by four mountings 100, 102, 104, 106, exemplified here. In further exemplary embodiments mentioned in the general description, more or fewer mountings may also be provided, or the design of the mountings may differ from the design shown in FIG. 2.

In use of the speaking valve 10, pressure is built up from the second side 92 when the user exhales, which de-places the sealing means 94 towards the first side 90. This deplacement is greater when the user is attempting to speak, as naturally the exhalation pressure is greater when speaking. When the cover part 50 is screwed onto the insertion part 30 to the extent that the sealing means 94 thereby comes into contact with the valve seat 57, the speaking valve 10 is closed and exhaled air can flow through the user's larynx or a shunt valve to allow the user to speak.

The proposed speaking valve 10 advantageously enables the user to speak handsfree. Furthermore, the user is comfortably given the possibility to adjust the speaking valve 10 to his needs or current situation and to adjust the necessary exhalation pressure to initiate speaking.

1. Speaking valve at least comprising:
a housing and a closing means, wherein the housing comprises an insertion part and a cover part with a breathing opening, wherein the cover part can be arranged on the insertion part, wherein the closing means comprises an outer ring, a sealing means and at least one mounting, wherein the sealing means is attached to the outer ring by means of the mounting, wherein the cover part forms a valve seat for the abutment of the sealing means and the breathing opening can be closed by means of the sealing means.

2. Speaking valve according to claim 1 wherein the at least one mounting is web-shaped.

3. Speaking valve according to claim 1 wherein by the mounting the sealing means is rubber-elastically mounted on the outer ring.

4. Speaking valve according to claim 1 the closing means is at least partially disposable in an at least partially circumferential closing means receptacle of the housing.

5. Speaking valve according to the insertion part comprises a threaded portion having a thread and the cover part comprises a cover thread, wherein the thread of the insertion part and the cover thread are configured to correspond with each other.

6. Speaking valve according to claim 5, wherein the thread and the cover thread have about 0.5 to about 2 turns.

7. Speaking valve according claim 5 wherein the thread and the cover thread is a metric thread pitch of about 0.5 to about 2 times a height of the threaded portion.

8. Speaking valve according to claim 1 wherein a distance of the closing means from the valve seat can be varied by turning the cover part on the insertion part.

9. Speaking valve according to claim 1 including a filter disposed in the insertion part, wherein the insertion part comprises a filter lock for-retaining the filter in the insertion part.

10. Closing means for a speaking valve according claim 1 comprising an outer ring, a sealing means and at least one mounting, wherein the sealing means is disposed on the outer ring by the mounting.

11. Closing means according to claim 10, wherein the at least one mounting is web-shaped.

12. Closing means according to claim 10 wherein by means of the mounting the sealing means is rubber-elastically mounted on the outer ring.

13. Method for adjusting a closing pressure of a speaking valve comprising the steps of:
providing a housing and a closing means, wherein the housing comprises an insertion part and a cover part with a breathing opening and a valve seat,
arranging the cover part on the insertion part, wherein the closing means comprises an outer ring, a sealing means and at least one mounting, wherein the sealing means is disposed on the outer ring by the mounting,
varying a distance between the sealing means and the valve seat by turning the cover part on the insertion part.
